(19) United States
(12) Patent Application Publication
(10) Pub. No.: US 2026/0056337 A1
(43) Pub. Date: Feb. 26, 2026

(54) METHOD FOR SENSITIVITY CALIBRATION

(71) Applicant: SHANGHAI UNITED IMAGING HEALTHCARE CO., LTD., Shanghai (CN)

(72) Inventors: Songsong TANG, Shanghai (CN); Bei'en WANG, Shanghai (CN); Junwei LI, Shanghai (CN); Yun DONG, Shanghai (CN)

(21) Appl. No.: 19/307,093

(22) Filed: Aug. 22, 2025

(30) Foreign Application Priority Data
Aug. 22, 2024 (CN) 202411164271.0

(51) Int. Cl. G01T 7/00; A61B 6/58; G01T 1/164

(52) U.S. Cl. CPC G01T 7/005; A61B 6/583; G01T 1/1642; G01T 1/1647

(57) ABSTRACT

A sensitivity calibration method includes: obtaining a mapping relationship between a plurality of groups of calibration parameters and a plurality of state variations of a target system; obtaining a first energy response during a first time period and a second energy response during a second time period; wherein the target system is in a first state during the first time period, and the target system is in a second state during the second time period; determining a state variation of the target system in the second state relative to the first state based on a comparison result of the first energy response and the second energy response; calibrating a sensitivity of the target system based on the mapping relationship between the plurality of groups of calibration parameters of the target system and the plurality of state variations of the target system and the current state variation of the target system.

### CROSS-REFERENCE TO RELATED APPLICATION

This application claims priority to Chinese Patent Application No. 202411164271.0, filed on Aug. 22, 2024, the entire contents of which are hereby incorporated by reference.

### TECHNICAL FIELD

The present disclosure relates to the field of medical imaging technology, and in particular, to methods, devices, and storage media for sensitivity calibration.

### BACKGROUND

Single Photon Emission Computed Tomography (SPECT) is a mature imaging technology in the field of nuclear medicine today, widely used in clinical detection. The sensitivity of a SPECT system is closely related to the accuracy of quantitative SPECT imaging.

In the related art, measurements are conducted on liquid or solid radioactive sources with known radioactivity levels, and the system sensitivity is time periodically calculated and calibrated based on the measurement results. However, as the actual sensitivity of the system changes with the temporal evolution of the state of the target system, the calibration effect of the aforementioned method is inadequate during the interval between two successive sensitivity calibrations. Specifically, the system sensitivity cannot be accurately calibrated to accommodate the continuous changes in the state of the target system.

Therefore, it is desirable to provide an effective method or device for calibrating sensitivity accurately to adapt to the continuous changes in the state of the target system.

### SUMMARY

An aspect of the present disclosure may provide A method for sensitivity calibration, the method includes: obtaining a mapping relationship between a plurality of groups of calibration parameters of a target system and a plurality of state variations of the target system; obtaining a first energy response of the target system during a first time period and a second energy response of the target system during a second time period; wherein the target system is in a first state during the first time period, and the target system is in a second state during the second time period; determining a state variation of the target system in the second state relative to the first state based on a comparison result of the first energy response and the second energy response; calibrating a sensitivity of the target system based on the mapping relationship between the plurality of groups of calibration parameters of the target system and the plurality of state variations of the target system and the current state variation of the target system.

In some embodiments, the obtaining the mapping relationship between the plurality of groups of calibration parameters of the target system and the plurality of state variations includes: obtaining multiple energy responses and multiple sensitivities of the target system under multiple different states of the target system respectively, wherein one of the multiple energy responses and one of the multiple sensitivities of the target system correspond to one of the multiple different states of the target system; determining the mapping relationship between the plurality of state calibration parameters and the plurality of state variations of the target system based on the multiple energy responses and the multiple sensitivities of the target system under the multiple different states of the target system.

In some embodiments, each of the multiple energy responses of the target system is represented by a preset function.

In some embodiments, the preset function includes a Gaussian function, a double Gaussian function, or a composite function composed of the Gaussian function and the exponential function.

In some embodiments, the mapping relationship is a relationship between variations of one or more target parameters in the preset function and the plurality of groups of calibration parameters.

In some embodiments, the mapping relationship between the plurality of groups of calibration parameters of the target system and the plurality of state variations of the target system includes: selecting the mapping relationship between the plurality of groups of calibration parameters of the target system and the plurality of state variations of the target system from a preset mapping relationship library based on a combination of isotope types and collimator types.

In some embodiments, the obtaining the first energy response of the target system during the first time period and the second energy response of the target system during the second time period includes: obtaining first scan data of the target system during the first time period, and determining the first energy response by performing superposition processing on the first scan data; obtaining second scan data of the target system during the second time period, and determining the second energy response by performing superposition processing on the second scan data.

In some embodiments, the calibrating the sensitivity of the target system based on the mapping relationship between the plurality of groups of calibration parameters of the target system and the plurality of state variations of the target system and the current state variation of the target system includes: determining one group of calibration parameters corresponding to the state variations of the target system based on the mapping relationship between the plurality of groups of calibration parameters of the target system and the plurality of state variations of the target system and the current state variation of the target system, wherein the one group calibration parameters include at least one of energy window parameters or scale factor.

In some embodiments, the calibrating the sensitivity of the target system based on the mapping relationship between the plurality of groups of calibration parameters of the target system and the plurality of state variations of the target system and the current state variation of the target system: calibrating an energy window of the target system based on the mapping relationship between the plurality of groups of calibration parameters of the target system and the plurality of state variations of the target system and the current state variation of the target system.

In some embodiments, he calibrating the sensitivity of the target system based on the mapping relationship between the plurality of groups of calibration parameters of the target system and the plurality of state variations of the target system and the current state variation of the target system includes: determining the energy window parameters based on the mapping relationship between the plurality of groups of calibration parameters of the target system and the plurality of state variations of the target system and the current state variation of the target system, wherein the energy window parameters at least include a center position of the energy window or a width of the energy window; calibrating the energy window of the target system based on the energy window parameters.

In some embodiments, the calibrating the sensitivity of the target system based on the mapping relationship between the plurality of groups of calibration parameters of the target system and the plurality of state variations of the target system and the current state variation of the target system includes: calibrating a count rate of the target system based on the mapping relationship between the plurality of groups of calibration parameters of the target system and the plurality of state variations of the target system and the current state variation of the target system.

In some embodiments, the calibrating the count rate of the target system based on the mapping relationship between the plurality of groups of calibration parameters of the target system and the plurality of state variations of the target system and the current state variation of the target system includes: determining a scale factor of the target system based on the mapping relationship between the plurality of groups of calibration parameters of the target system and the plurality of state variations of the target system and the current state variation of the target system, when an energy window corresponding to the first state and an energy window corresponding to the second state of the target system are the same; calibrating the count rate of the target system based on the scale factor of the target system.

In some embodiments, the determining the scale factor of the target system based on the mapping relationship between the plurality of groups of calibration parameters of the target system and the plurality of state variations of the target system and the current state variation of the target system includes: constructing a mapping relationship between multiple energy drifts and multiple scale factors; determining the scale factor corresponding to the current state variation of the target system based on the mapping relationship between the multiple energy drifts and multiple scale factors.

Another aspect of the present disclosure may provide a method for sensitivity calibration, the method include: obtaining an energy response of a target system during a calibrated time period; obtaining a reference energy response of the target system; determining whether an energy variation between the energy response during the calibrated time period and the reference energy response is greater than an energy threshold; if the energy variation is greater than the energy threshold, calibrating the sensitivity of the target system based on the variation of the energy response during the calibrated time period relative to the reference energy response, wherein the calibrating the sensitivity of the target system includes at least one of calibrating an energy window of the target system or calibrating a count rate of the target system.

In some embodiments, the calibrating the sensitivity of the target system includes: determining a current state of the target system during the calibrated time period and a reference state of the target system, wherein the reference state corresponds to the reference energy response; determining a current state variation of the target system relative to the reference state of the target system based on a comparison result of the energy response during the calibrated time period and the reference energy response; determining a mapping relationship between a plurality of groups of calibration parameters of the target system and a plurality of state variations of the target system; calibrating the sensitivity of the target system based on the mapping relationship between the plurality of groups of calibration parameters of the target system and the plurality of state variations of the target system and the current state variation of the target system, wherein the calibrating the sensitivity of the target system includes at least one of calibrating the energy window of the target system or calibrating the count rate of the target system.

In some embodiments, the calibrating the sensitivity of the target system includes at least one of the energy window of the target system or the count rate of the target system based on the mapping relationship the plurality of groups of calibration parameters of the target system and the plurality of state variations of the target system and the current state variation of the target system includes: determining energy window parameters based on the mapping relationship between the plurality of groups of calibration parameters of the target system and the plurality of state variations of the target system and the current state variation of the target system, wherein the energy window parameters include at least one of a center position of the energy window or a width of the energy window; calibrating the energy window of the target system based on the energy window parameters.

In some embodiments, the calibrating the sensitivity of the target system includes at least one of the energy window of the target system or the count rate of the target system based on the mapping relationship between the plurality of groups of calibration parameters of the target system and the plurality of state variations of the target system and the current state variation of the target system includes: determining a scale factor of the target system based on the mapping relationship between the plurality of groups of calibration parameters of the target system and the plurality of state variations of the target system and the current state variation of the target system, when an energy window corresponding to the current state and an energy window corresponding to the reference state of the target system are the same; calibrating the count rate of the target system based on the scale factor of the target system.

In some embodiments, the determining the scale factor of the target system based on the mapping relationship between the plurality of groups of calibration parameters of the target system and the plurality of state variations of the target system and the current state variation of the target system includes: constructing a mapping relationship between multiple energy drifts and the multiple scale factors; determining the scale factor of the target system corresponding to the current state variation of the target system based on the mapping relationship between the multiple energy drifts and the multiple scale factors.

In some embodiments, the method further includes: if the energy variation between the energy response during the calibrated time period and the reference energy response is less than or equal to the energy threshold, taking the sensitivity of the target system in the reference state as the sensitivity of the target system in the current state.

Another aspect of the present disclosure may provide a method for sensitivity calibration, the method include: obtaining multiple sets of scan data of a target system according to a first condition, wherein the first condition at least includes satisfying a preset time or a number of scans reaching a first threshold; determining an energy response curve of the target system based on the multiple sets of scan data; determining whether it is necessary to calibrate the sensitivity of the target system based on the energy response curve of the target system.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further illustrated in terms of exemplary embodiments. These exemplary embodiments are described in detail with reference to according to the drawings. These embodiments are non-limiting exemplary embodiments, in which like reference numerals represent similar structures.

FIG. 1 is a schematic diagram illustrating an exemplary a SPECT system according to some embodiments of the present disclosure.

FIG. 2 is a schematic diagram illustrating exemplary modules of a sensitivity calibration device of a SPECT system according to some embodiments of the present disclosure.

FIG. 3 is a flowchart of a sensitivity calibration method of a SPECT system according to some embodiments of the present disclosure.

FIG. 4 is a schematic diagram illustrating exemplary energy responses in different states according to some embodiments of the present disclosure.

FIG. 5 is a schematic diagram illustrating an exemplary energy responses of a sensitivity calibration phantom and an average human body according to some embodiments of the present disclosure.

FIG. 6 is a schematic diagram illustrating an exemplary a preset mapping relationship library according to some embodiments of the present disclosure.

FIG. 7 is a schematic diagram illustrating exemplary energy window settings in different state of the target systems according to some embodiments of the present disclosure.

FIG. 8 is a schematic diagram illustrating exemplary energy responses in different state of the target systems under the same energy window according to some embodiments of the present disclosure.

FIG. 9 is a schematic diagram illustrating an exemplary a mapping relationship between a scale factor and energy drift according to some embodiments of the present disclosure.

FIG. 10 is a flowchart of a sensitivity calibration method of a SPECT system according to some embodiments of the present disclosure.

FIG. 11 is a flowchart of a sensitivity calibration method of a SPECT system according to some embodiments of the present disclosure.

FIG. 12 is a schematic diagram illustrating exemplary modules of a sensitivity calibration device of a SPECT system according to some embodiments of the present disclosure.

### DETAILED DESCRIPTION OF THE EMBODIMENT

In the following detailed description, numerous specific details are set forth by way of examples in order to provide a thorough understanding of the relevant disclosure. However, it should be apparent to those skilled in the art that the present disclosure may be practiced without such details.

In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but is to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “include,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the terms “system,” “engine,” “unit,” “module,” and/or “block” used herein are one method to distinguish different components, elements, parts, sections or assemblies of different levels in ascending order. However, the terms may be displaced by another expression if they achieve the same purpose.

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device.

Software instructions may be embedded in firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that when a unit, engine, module, or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items. The terms “pixel” and “voxel” in the present disclosure are used interchangeably to refer to an element of an image.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

Compared with related art, the method for sensitivity calibration and computer device for the SPECT system and the computer device provided in this embodiment obtain a mapping relationship between calibration parameters of a target system and state of the target system variations; obtain a first energy response of the target system during a first time period and a second energy response of the target system during a second time period; determine a current state of the target system variation of the second state relative to the first state based on a comparison result of the first energy response and the second energy response; calibrate a sensitivity of the target system based on the mapping relationship between the calibration parameters of the target system and the current state of the target system variation. This solves the problem that system sensitivity cannot be accurately calibrated to adapt to continuous changes of the state of the target system, achieves accurate calibration of the system sensitivity, and ensures that the calibrated system is adapted to continuous changes of the state of the target system.

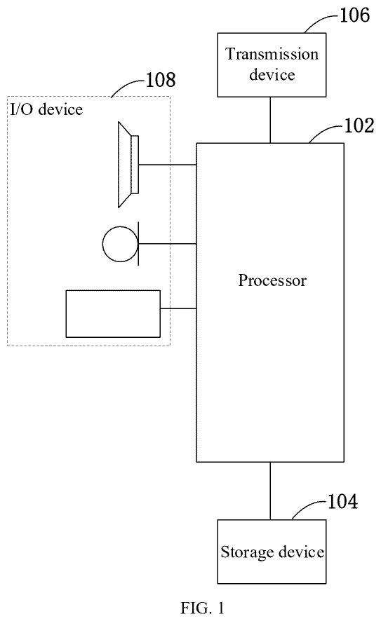

FIG. 1 is a schematic diagram illustrating an exemplary a SPECT system according to some embodiments of the present disclosure. As shown in FIG. 1, a SPECT system 100 includes a processor 102, a storage device 104, a transmission device 106, and an input/output device 108. The processor 102 may include but is not limited to a processing device such as a microprocessor MCU or a programmable logic device FPGA. The terminal may further include a transmission device 106 for communication functions and an input/output device 108.

The storage device 104 can be used to store computer programs, such as software program and modules of application software, for example, the computer program corresponding to the sensitivity calibration method of the SPECT system in the present embodiment. The processor 102 executes various functional applications and data processing by running the computer program stored in the storage device 104, that is, implementing the above method. The storage device 104 may include a high-speed random access memory, and may also include a non-volatile memory, such as one or more magnetic storage devices, flash memory, or other non-volatile solid-state memories. In some examples, the storage device 104 may further include memories remotely disposed relative to the processor 102, and these remote memories may be connected to the terminal through a network.

Examples of the above network include, but are not limited to, the Internet, an enterprise intranet, a local area network, a mobile communication network, and combinations thereof.

The transmission device 106 is used to receive or send data via a network. The above network includes a wireless network provided by a communication provider of the terminal. In one example, the transmission device 106 includes a network interface controller (NIC) which can be connected to other network devices through a base station to communicate with the Internet. In one example, the transmission device 106 may be a radio frequency (RF) module for communicating with the Internet in a wireless manner.

It should be noted that the descriptions of the medical system 100 are provided for illustrative purposes only and not intended to limit the scope of the present disclosure. For a person of ordinary skill in the art, a variety of modifications or variations may be made according to the description of the present disclosure. In some embodiments, the medical system 100 may be implemented on other devices to achieve similar or same functions. In some embodiments, the medical system 100 may include one or more additional components and may omit one or more of the components. Additionally or alternatively, two or more components of the medical system 100 may be integrated into a single component. As another example, the components of the medical system 100 may be replaced by another component that may fulfill the functions of the components. However, these variations and modifications do not depart from the scope of the present disclosure.

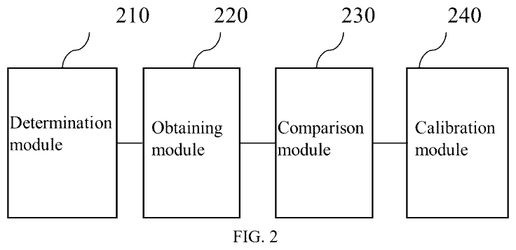

FIG. 2 is a schematic diagram illustrating exemplary modules of a SPECT system according to some embodiments of the present disclosure. In some embodiments, a medical system 200 includes a determination module 210, an obtaining module 220, comparison module 230, and a calibration module 240.

The determination module 210 may be configured to obtaining a mapping relationship between a plurality of groups of calibration parameters of a target system and a plurality of state variations of the target system. More descriptions regarding determination module 210 may be found in operation 310 and are not repeated here.

The obtaining module 220 may be configured to obtaining a first energy response of the target system during a first time period and a second energy response of the target system during a second time period; wherein the target system is in a first state during the first time period, and the target system is in a second state during the second time period. More descriptions regarding obtaining module 220 may be found in operation 320 and are not repeated here.

The comparison module 230 may be configured to determining a state variation of the target system in the second state relative to the first state based on a comparison result of the first energy response and the second energy response. More descriptions regarding comparison module 230 may be found in operation 330 and are not repeated here.

The calibration module 240 may be configured to calibrating a sensitivity of the target system based on the mapping relationship between the plurality of groups of calibration parameters of the target system and the plurality of state variations of the target system and the current state variation of the target system. More descriptions regarding calibration module 240 may be found in operation 330 and are not repeated here.

It is to be noted that the above descriptions of the medical system 200 are provided for illustration purposes, and do not limit the present disclosure to the scope of the cited embodiments. It is to be understood that for a person skilled in the art, with an understanding of the principle of the system, it may be possible to arbitrarily combine the modules or form subsystems that are connected to other modules without departing from this principle. In some embodiments, the acquisition module 210, the determination module 220, and the reconstruction module 230 disclosed in FIG. 2 may be different modules in a system, or one module realizing the functions of two or more other modules.

For example, the modules may share a common storage module, and the modules may each have a respective storage module. Such deformations are within the scope of protection of the present disclosure.

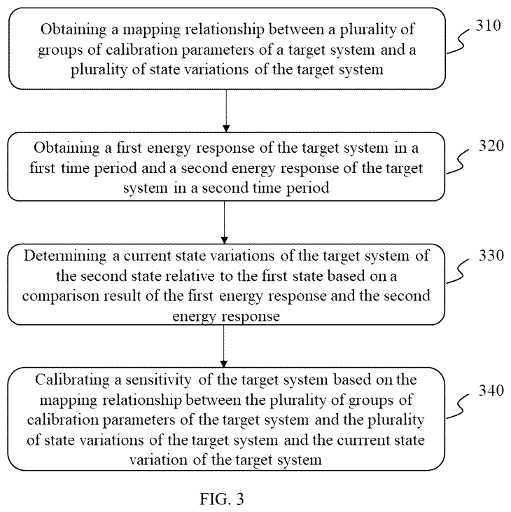

FIG. 3 is a flowchart illustrating an exemplary a sensitivity calibration method of a SPECT system according to some embodiments of the present disclosure; In some embodiments, process 300 may be performed by the processor 102. For example, the process 300 may be stored in the form of instructions in the storage device 104 and the process 300 may be implemented when the processor 102 or the medical system 200 executes the instructions.

In 310, obtain a mapping relationship between a plurality of groups of calibration parameters of a target system and a plurality of state variations of the target system. In some embodiments, operation 310 may be performed by the determination module 210.

In some embodiments, the state of the target system of the target system is changed by changing the detector state of the target system. For example, the detector state of the target system can be changed by setting different environmental temperatures, humidity, and dust conditions. Under multiple different states of the target system respectively, one of the multiple energy responses and one of the multiple sensitivities of the target system correspond to one of the multiple different states of the target system are obtained, and determining the mapping relationship between the plurality of state calibration parameters and the plurality of state variations of the target system based on the multiple energy responses and the multiple sensitivities of the target system under the multiple different states of the target system, wherein the calibration parameters are used to calibrate the sensitivity of the target system.

In some embodiments, one group calibration parameters include at least one of energy window parameters or scale factor.

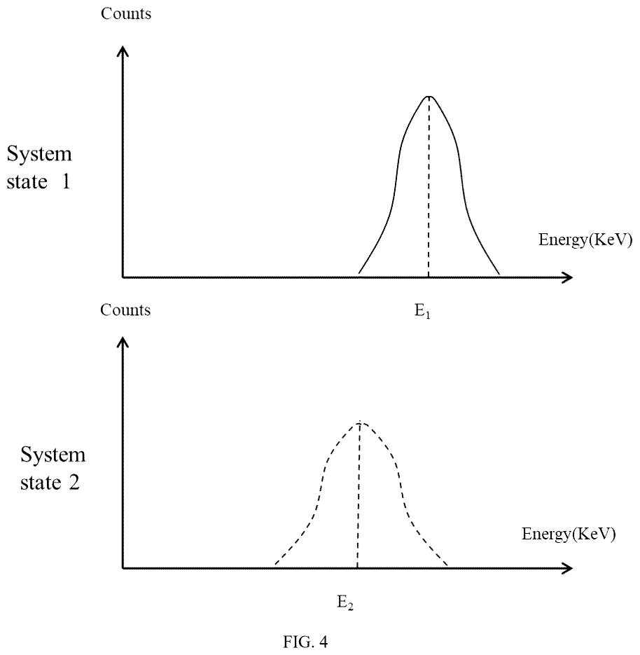

In some embodiments, an average energy response of the human body can reflect the variation of the state of the target system. As shown in FIG. 4, (a) and (b) in FIG. 4 are the energy response curves of the target system in system state 1 and system state 2, respectively. The abscissa represents the photon energy, and the vertical coordinate represents the number of photons received by the statistical detector under different energy distributions. The count rate of the SPECT system refers to the number of energy events recorded by the detector within a certain time period of time. When the count rate of the system changes, the number of photons received by the detector changes accordingly. E1 and E2 are the energy peaks in system state 1 and system state 2, respectively. E1 represents the energy peaks of the target system in the second time period when it is in the second state. State 1 refers to the first state of the target system in the first time period, and state 2 refers to the second state of the target system in the second time period. The energy peak is the energy where the distribution of the number of photons collected by the detector is the most concentrated under an isotope with known activity. An isotope usually corresponds to one or more energy peaks (for example, the energy peak of 99mTc3 is 141 KeV, and the energy peak of 131I7 is 364 keV). Under different state of the target systems, the average energy response of the human body will change accordingly. In some embodiments, the energy response in this state of the target system of the target system is represented by an energy histogram.

In some embodiments, the superposition of multiple sets of scan data of the SPECT system includes scan data of human body, and a specific isotope usually corresponds to one or more energy peaks. The energy distribution after superposing multiple sets of scan data can be approximately the detector's average energy response of the human body, such as superposing scan data of the same part of multiple scanned objects, or superposing scan data of scanned objects with similar body mass indexes, etc. The superposition method includes statistical Gaussian distribution of scan data. Utilize the characteristics of the Gaussian distribution to perform superposition processing on multiple sets of scan data.

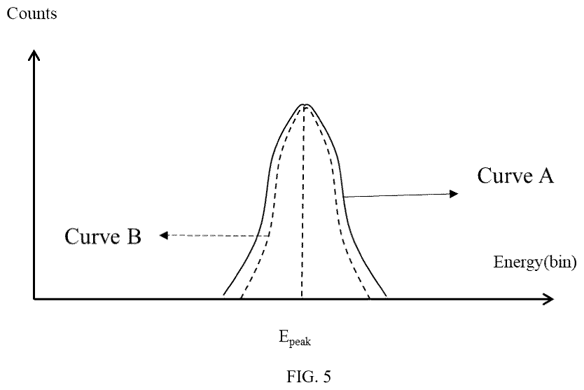

In some embodiments, a water phantom simulating the human body or a cylindrical uniform water phantom is used as the sensitivity calibration phantom, so that the energy response of the target system's detector to the sensitivity calibration phantom is close to detector's average energy response of the human body. As shown in FIG. 5, curve A and curve B respectively represent the energy response of the target system's detector to the sensitivity calibration phantom and the detector's average energy response of the human body. The energy peak of curve A is basically the same as that of curve B, which is Epeak.

In some embodiments, the method for determining and constructing the mapping relationship includes measuring energy responses and sensitivities using a sensitivity calibration phantom, so that the energy response of the system detector to the sensitivity calibration phantom is close to its average energy response of the human body, thereby being able to reflect the state variation of the target system through the energy response of the system, while ensuring the accuracy of obtaining energy responses and sensitivities.

In 320, obtain a first energy response of the target system during a first time period and a second energy response of the target system during a second time period; wherein the target system is in a first state during the first time period, and the target system is in a second state during the second time period. In some embodiments, operation 320 may be performed by the obtaining module 220.

In some embodiments, in the initial stage of use of the target system (for example, when the system is newly installed or the first sensitivity calibration after a system failure), the sensitivity of the system is calibrated in advance using a sensitivity calibration phantom, and this sensitivity of the target system can be used as a reference for subsequent sensitivity calibration. Multiple sets of data actually scanned by the target system under different state of the target systems of the target system is accumulated to facilitate obtaining energy responses of the target system under different states.

In some embodiments, during the actual scanning procedure of the target system, first, first scan data of the target system within a first time period is obtained, where the first scan data includes multiple clinical data corresponding to multiple scans of the target system within the first time period. Superposition processing is performed on the first scan data to obtain a first energy response. When the system is in a second time period, the second scan data of the target system is obtained, where the second scan data includes clinical data corresponding to multiple scans of the target system within the second time period. Superposition processing is performed on the second scan data to obtain a second energy response. For example, the target system is in a first state within the first time period and in a second state within the second time period, and the first time period and the second time period may correspond to a historical time period and a current time period, respectively. In some embodiments, the first state is the average state of the state of the target system of the target system within the first time period, and the second state is the average state of the state of the target system of the target system within the second time period.

In 330, determining a state variation of the target system in the second state relative to the first state based on a comparison result of the first energy response and the second energy response. In some embodiments, operation 330 may be performed by the comparison module 230.

In some embodiments, with the first energy response as a reference energy response, the second energy response is compared with the reference energy response to obtain the energy response variation of the target system between different states of the target system, which reflects the state of the target system change of the target system, so that the clinical data scanned by the target system can be used to calibrate and monitor the state of the target system variation, so as to determine the current state of the target system variation of the second state relative to the first state, that is, the change of the target state of the target system in the second time period compared with that in the first time period. According to the actual state change of the target system and the mapping relationship, the system sensitivity is corrected accordingly, which solves the problem that the system sensitivity cannot be accurately calibrated to adapt to the continuous change of the state of the target system, realizes a significant extension of the calibration cycle, accurately calibrates the system sensitivity, ensures that the calibrated system adapts to the continuous change of the state of the target system, and provides the accuracy of system sensitivity calibration.

In 340, calibrate a sensitivity of the target system based on the mapping relationship between the calibration parameters of the target system and the current state of the target system variation. In some embodiments, operation 340 may be performed by the calibration module 240.

In some embodiments, according to the relationship between the plurality of groups of calibration parameters of the target system and the plurality of state variations of the target system and the current state variation of the target system, current groups of calibration parameters corresponding to the current state of the target system variation are determined, and the current calibration parameters are used to calibrate the sensitivity of the target system.

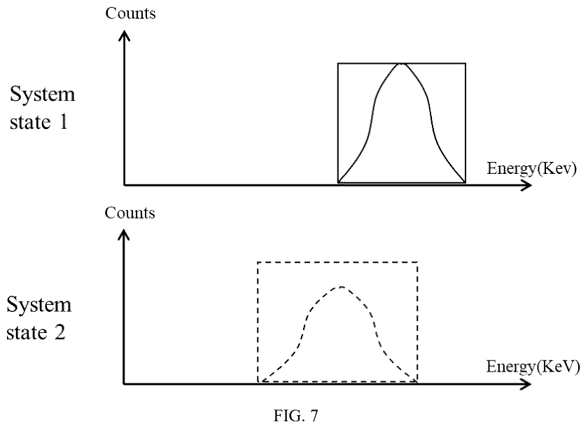

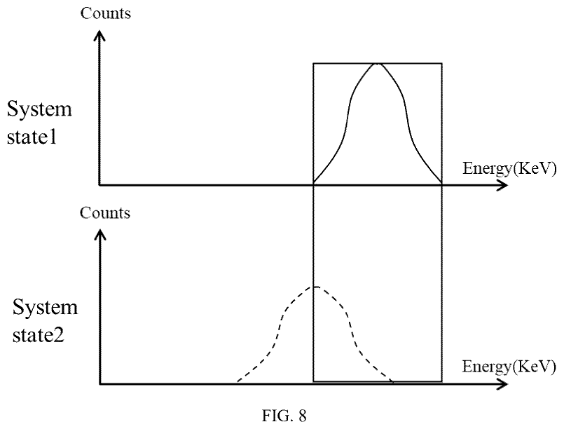

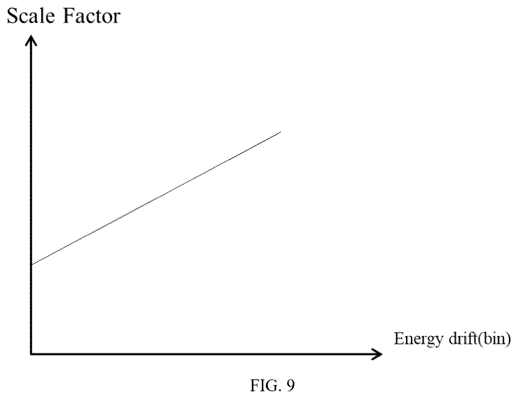

In some embodiments, the groups of calibration parameters include at least one of an energy window parameter of the target system and a scale factor of the target system. The above sensitivity calibration the target system includes at least one of calibrating the energy window based on the energy window parameter or calibrating the count rate based on the scaling factor. In some embodiments, calibrating the energy window based on the energy window parameter includes adjusting the center position of the energy window or the width of the energy window, etc.; more descriptions about calibrating the energy window based on the energy window parameter can be found in other parts of the present disclosure (for example, FIG. 7 and related descriptions). When calibrating the count rate based on the scaling factor, on the basis of keeping the energy window unchanged, the count rate of the target system is calibrated by setting a reasonable scaling factor. More descriptions about calibrating the count rate based on the scaling factor can be found in other parts of the present disclosure (for example, FIG. 8, FIG. 9 and related descriptions).

SPECT quantification refers to the use of Single-Photon Emission Computed Tomography (SPECT) technology to quantitatively analyze the distribution of radioactive tracers in specific organs or tissues in the body, and to achieve objective evaluation, condition monitoring and efficacy judgment of diseases by calculating specific values such as radioactive activity, uptake rate, and metabolic rate.

To achieve the accuracy of quantitative analysis of SPECT images, measurements are usually performed on liquid or solid radioactive sources with known radioactive activity, and the system sensitivity is calculated and calibrated regularly based on the measurement results. However, because the state of the target system changes continuously over time, the actual sensitivity of the system will also change continuously. For the time period between two sensitivity calibrations, if the sensitivity after the last calibration is used as the reference to calibrate the current system sensitivity, the calibration effect will gradually deteriorate, that is, it cannot adapt to the continuous change of the state of the target system, reducing the accuracy of system sensitivity calibration.

Compared with the related art, the present application determines the mapping relationship between the plurality of groups of calibration parameters of the target system and; obtaining a first energy response of the target system during a first time period and a second energy response of the target system during a second time period; wherein the target system is in a first state during the first time period, and the target system is in a second state during the second time period; determining a state variation of the target system in the second state relative to the first state based on a comparison result of the first energy response and the second energy response; and further calibrates the sensitivity of the target system based on the mapping relationship between the plurality of groups of calibration parameters of the target system and the plurality of state variations of the target system. Based on this, the energy response of the system detector is used to reflect the state of the target system variation to establish a mapping relationship between a plurality of groups of calibration parameters of a target system and a plurality of state variations of the target system, and the sensitivity of the target system is corrected according to the actual state change of the target system and the mapping relationship between the plurality of groups of calibration parameters of the target system and the plurality of state variations of the target system, which solves the problem that the system sensitivity cannot be accurately calibrated to adapt to the continuous change of the state of the target system, realizes a significant extension of the calibration cycle, accurately calibrates the system sensitivity, and ensures that the calibrated system adapts to the continuous change of the state of the target system.

In some of these embodiments, determining the mapping relationship between the plurality of groups of calibration parameters of the target system and the plurality of state variations of the target system in step 210 includes Step 211 to Step 212.

Step 211, obtain multiple energy responses and multiple sensitivities of the target system under multiple different states of the target system respectively, wherein one of the multiple energy responses and one of the multiple sensitivities of the target system correspond to one of the multiple different states of the target system; Sensitivity of the target system refers to the counts recorded by a detector per unit time per unit activity of a radioactive source (expressed as counts/(s-MBq)), which reflects the overall detection capability of the system. In some embodiments, the sensitivity of the current target system is calculated by actually measuring the photon counting rate received by the detector of the target system using a radioactive source with known activity, and combining it with the activity of the radioactive source with known activity. For example, the formula for calculating sensitivity can be expressed as: Sensitivity=(Total counts−Background counts)/(Activity*Acquisition time).

Step 212, determining the mapping relationship between the plurality of state calibration parameters and the plurality of state variations of the target system based on the multiple energy responses and the multiple sensitivities of the target system under the multiple different states of the target system.

In some embodiments, the state of the target system of the target system is changed by setting different environmental conditions, etc. Under different state of the target systems, the sensitivity is measured multiple times using a sensitivity calibration phantom, and the energy responses of the target system under various state of the target systems are obtained at the same time. The energy response of the system detector to the sensitivity calibration phantom is close to the average energy response of the human body under the same state of the target system.

In some embodiments, the mean, variance, or standard deviation of multiple sensitivities under each state of the target system is calculated as the sensitivity corresponding to the state of the target system, so as to ensure the accuracy of sensitivity measurement. Since the change in the average energy response of the human body can reflect the change in the state of the target system, the mapping relationship between the plurality of groups of calibration parameters of the target system and the plurality of state variations of the target system is constructed according to the sensitivity change and energy response change of the target system under different state of the target systems, and the calibration parameters are used to calibrate the sensitivity of the target system.

In the above embodiment, the energy responses and sensitivities of the target system are obtained, and the mapping relationship between the plurality of groups of calibration parameters of the target system is accurately constructed based on the multiple energy responses and multiple sensitivities of the target system.

In some of these embodiments, the energy response of the target system is represented by a preset function. The energy response of the system detector to the average human body is used to reflect the state of the target system variation, and the energy response of the detector to the average human body can be simplified as a preset function. In some embodiments, the preset function includes a Gaussian function, a double Gaussian function, a composite function composed of a Gaussian function and an exponential function, etc.

In some embodiments, according to the energy response curve of the detector to the average human body under each state of the target system, the function formula corresponding to the state of the target system is determined, so as to parameterize the multiple different states of the target system.

For example, as shown in FIG. 4, the energy responses in system state 1 and system state 2 are represented by Gaussian functions. The energy response in system state 1 is represented according to formula (1):

$$P ( E ) = \frac{A}{\sqrt{2 πσ}} exp [ - \frac{{( E - μ )}^{2}}{2 {σ}^{2}} ] \qquad ( 1 )$$

In formula (1), A represents the peak value of the energy response curves of the target system; a represents the standard deviation of energy, and represents the mean value of energy, respectively.

The energy response in system state 2 is represented according to formula (2):

$${P}^{′} ( E ) = \frac{{A}^{′}}{\sqrt{2 {πσ}^{′}}} exp [ - \frac{{( E - {μ}^{′} )}^{2}}{2 {σ}^{′2}} ] \qquad ( 2 )$$

In formula (2), A′ represents the peak value of the energy response curves of the target system; σ′ represents the standard deviation of energy, and σ′ represents the mean value of energy, respectively.

In this embodiment, each of the multiple energy responses of the target system is represented by a preset function so as to parameterize the system state and facilitate the establishment of the mapping relationship between the plurality of groups of calibration parameters of the target system and the plurality of state variations of the target system.

In some of these embodiments, the mapping relationship is a relationship between variations of one or more target parameters in the preset function and the plurality of groups of calibration parameters.

In some embodiments, the preset function is used to represent the average energy response of the human body of the detector under each one of the multiple different states of the target system so as to obtain the function formula corresponding to the state of the target system. The variation of each parameter in the formula reflects the state variation of the target system.

In some embodiments, the Gaussian function is used to represent the multiple energy responses of the detectors of the target system. Under a set state of the target system (for example, the ambient temperature is 25° C. and the relative humidity is 50%), the energy response is

$$P ( E ) = \frac{A}{\sqrt{2 πσ}} exp [ - \frac{{( E - μ )}^{2}}{2 {σ}^{2}} ] ,$$

where the target parameters in the formula include the curve peak A, the energy standard deviation and the energy mean value. The changes of the target parameters A can reflect the system state variation. Based on this, the mapping relationship between the system state variation and the calibration parameters is obtained by constructing the mapping relationship between the changes of tone or more target parameters in the preset function and the plurality of groups of calibration parameters.

Through this embodiment, the mapping relationship between the changes of tone or more target parameters in the preset function and the plurality of groups of calibration parameters is constructed, so as to accurately obtain the mapping relationship between the plurality of groups of calibration parameters of the target system and the plurality of state variations of the target system.

In some of these embodiments, determining the mapping relationship between the between the plurality of groups of calibration parameters of the target system and the plurality of state variations of the target system in Step 210 includes the following steps: selecting the mapping relationship between the plurality of groups of calibration parameters of the target system and the plurality of state variations of the target system from a preset mapping relationship library based on a combination of isotope types and collimator types.

Since the present embodiment approximates the energy distribution after superposing a large amount of clinical scan data as the energy response of the detector to the average human body, the average energy response of the human body of the detector is associated with the multiple isotope types and multiple collimator types. Based on different combinations of isotope and collimators, different mapping relationships can be constructed. Collimators include but are not limited to: parallel hole collimators, fan-beam collimators, and cone-beam collimators, etc. The set isotopes can be determined according to the isotopes to be used by the imaging system in practical applications.

In some embodiments, referring to the above method for mapping relationship between the plurality of groups of calibration parameters of the target system and the plurality of state variations of the target system, for different combinations of multiple isotopes and multiple collimators, corresponding mapping relationship between the plurality of groups of calibration parameters of the target system and the plurality of state variations of the target system are respectively constructed to establish a mapping relationship library, and different combinations of isotopes and collimators are stored corresponding to the mapping relationships. In practical applications, according to the multiple isotopes and multiple collimators in the target system, the corresponding mapping relationship is selected from the preset mapping relationship library as the mapping relationship between the plurality of groups of calibration parameters of the target system and the plurality of state variations of the target system and the current state variation of the target system. FIG. 5 is a structural schematic diagram of a preset mapping relationship library in an embodiment of the present application.

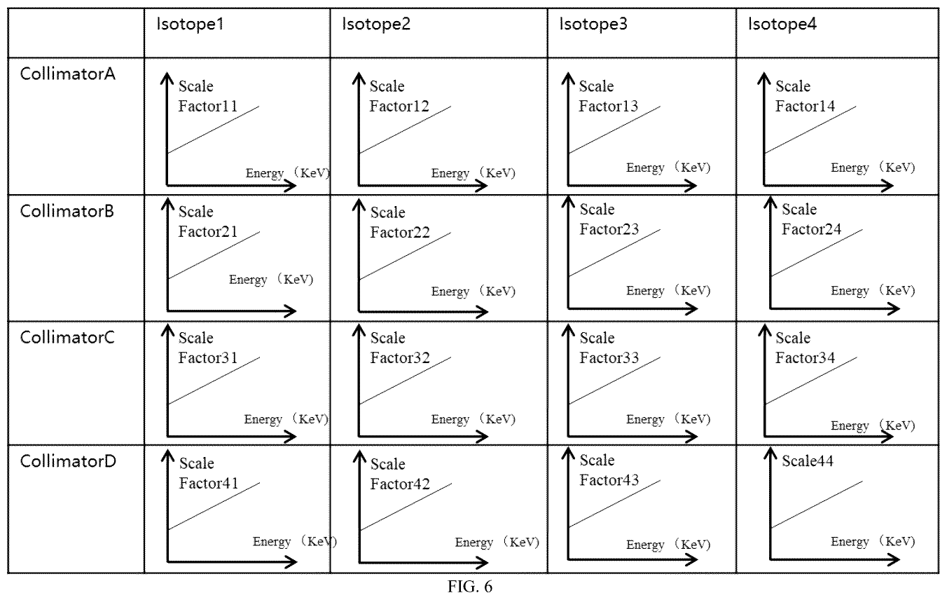

As shown in FIG. 6, in this embodiment, taking the calibration parameter as a scale factor as an example, for different combinations of multiple isotopes and multiple collimator, there are different mapping relationships between multiple scale factors and multiple state variations of the target system. In practical applications, the corresponding mapping relationship between multiple energy drifts and multiple scale factors is obtained from the preset mapping relationship library according to the multiple isotopes and multiple collimators used by the target system, as the mapping relationship between the plurality of groups of calibration parameters of the target system and the plurality of state variations of the target system.

Through this embodiment, according to multiple isotopes and multiple collimators in the target system, the mapping relationship between the calibration parameters and the system state variation is selected from the preset mapping relationship library, which avoids repeatedly executing the mapping relationship construction process for similar or identical systems, reduces the calibration cost, and improves the efficiency of presetting the mapping relationship mapping relationship between the plurality of groups of calibration parameters of the target system and the plurality of state variations of the target system.

In some of these embodiments, obtaining the first energy response of the target system within the first time period and the second energy response of the target system within the second time period in Step 220 includes Step 221 to Step 222.

Step 221, obtaining first scan data of the target system during the first time period, and determining the first energy response by performing superposition processing on the first scan data;

Step 222, obtaining second scan data of the target system during the second time period, and determining the second energy response by performing superposition processing on the second scan data.

In some embodiments, the first scan data of the target system within the first time period is obtained, where the first scan data includes a large amount of clinical data corresponding to multiple scans of the target system within the first time period. Superposition processing is performed on the energy information in the first scan data, and the energy distribution after superposition is the first energy response.

In some embodiments, when the target system is in the second time period, second scan data of the target system is obtained, where the second scan data includes a large amount of clinical data corresponding to multiple scans of the target system within the second time period. Superposition processing is performed on the energy information in the second scan data, and the energy distribution after superposition is the second energy response. The first time period and the second time period may be a historical period and a current period, respectively.

In some embodiments, the first scan data within a week is superposed to obtain the energy distribution after superposition as the first energy response, and the second scan data accumulated in the subsequent week is superposed to obtain the energy distribution after superposition as the second energy response.

In some embodiments, the scan data can be accumulated based on a preset number of scans, such as accumulating data every 50 scans. This embodiment does not limit the accumulation mode of clinical scan data.

Through this embodiment, the first scan data of the target system within the first time period is obtained, the first energy response is obtained by superposing the first scan data, and the second scan data of the target system within the second time period is obtained, and the second energy response is obtained by superposing the second scan data, so as to obtain the energy responses of the detector under different states, so as to accurately reflect the change of the system state, and ensure the objectivity of using the energy response change to reflect the change of the system state.

In some embodiments, calibrate sensitivity of the target system based on the mapping relationship between the plurality of groups of calibration parameters of the target system and the plurality of state variations of the target system and the current state variation of the target system. in Step 240 includes Step 241 to Step 242.

Step 241, calibrating an energy window of the target system based on the mapping relationship between the plurality of groups of calibration parameters of the target system and the plurality of state variations of the target system and the current state variation of the target system.

Step 242, or, calibrating a count rate of the target system based on the mapping relationship between the plurality of groups of calibration parameters of the target system and the plurality of state variations of the target system and the current state variation of the target system.

In some embodiments, the energy window of the SPECT system is a window for selecting the energy range of gamma rays received by the detector, and the setting mode of the energy window will change the sensitivity of the system. After determining the current system state variation, according to the pre-constructed mapping relationship between the plurality of groups of calibration parameters of the target system and the plurality of state variations of the target system, the current calibration parameters corresponding to the current system state variation are determined. The current calibration parameters include energy window parameters, which include the center position of the energy window, the width of the energy window, etc. The energy window of the target system is adjusted by setting the center position of the energy window or the width of the energy window, so that the number of photons received by the detector after the target system state changes remains basically unchanged, and the accuracy of sensitivity calibration is improved. As shown in FIG. 7, FIG. 7 is the energy window setting in system state 1, and (b) is the energy window setting in system state 2. When the system state changes from system state 1 to system state 2, or from system state 2 to system state 1, the energy window setting is adjusted accordingly through the energy window parameters to calibrate the sensitivity of the target system.

In some embodiments, the count rate of the SPECT system refers to the number of energy events recorded by the detector within a certain period of time. When the count rate of the system changes, the number of photons received by the detector changes accordingly, so that the sensitivity of the target system also changes accordingly. Based on this, according to the pre-constructed mapping relationship between the plurality of groups of calibration parameters of the target system and the plurality of state variations of the target system and the current system state variation, the current calibration parameters corresponding to the current system state variation are determined. On the basis of keeping the energy window unchanged, the scaling factor (Scale Factor) is calibrated according to the current calibration parameters, so as to set a reasonable Scale factor and realize the calibration of the count rate of the target system.

In some embodiments, for the calibration of the count rate, it is necessary to pre-construct a mapping relationship between energy drift and the Scale factor, as shown in FIG. 8. When the system state changes, according to the mapping relationship between energy drift and the Scale factor, the Scale factor under the energy response corresponding to the system state is selected to realize the calibration of the count rate. As shown in FIG. 8, the energy window setting in state 1 is the same as that in state 2. When the system state changes from system state 1 to system state 2, the number of photons received by the detector under the same energy window is reduced by half. According to the mapping relationship between multiple energy drifts and multiple scale factors, the Scale factor 2 corresponding to the system state is selected to calibrate the count rate, so as to realize the calibration of system sensitivity. In some embodiments, when system state 2 changes to state 1, the Scale factor is adjusted accordingly to calibrate the count rate. Under the same energy window, the number of photons received by the detector doubles. According to the mapping relationship between multiple energy drifts and multiple scale factors, the scale factor 0.5 corresponding to the system state is selected to calibrate the count rate, so as to realize the calibration of system sensitivity. the target system is in a first state during the first time period, and the target system is in a second state during the second time period.

Through the above embodiments, the energy window of the target system is calibrated based on the mapping relationship and the current system state variation, or the count rate of the target system is calibrated based on the mapping relationship and the current system state variation, so as to flexibly realize the calibration of system sensitivity, and thus the optimal way can be adopted to calibrate the target system.

The present embodiment also provides a sensitivity calibration method for a SPECT system.

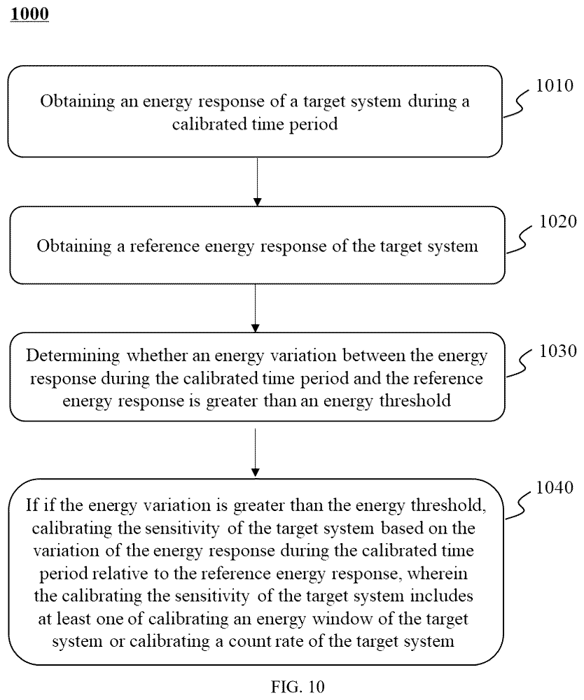

FIG. 10 is a flowchart of the sensitivity calibration method for the SPECT system in the present embodiment. As shown in FIG. 1-, the process includes the following step 1010 to step 1040.
Step 1010, determining a current state of the target system during the calibrated time period and a reference state of the target system, wherein the reference state corresponds to the reference energy response;
Step 1020, determining a current state variation of the target system relative to the reference state of the target system based on a comparison result of the energy response during the calibrated time period and the reference energy response;
Step 1030, determining a mapping relationship between a plurality of groups of calibration parameters of the target system and a plurality of state variations of the target system;
Step 1040, calibrating the sensitivity of the target system based on the mapping relationship between the plurality of groups of calibration parameters of the target system and the plurality of state variations of the target system and the current state variation of the target system, wherein the calibrating the sensitivity of the target system includes at least one of calibrating the energy window of the target system or calibrating the count rate of the target system.

In some embodiments, multiple scan data of the target system within the calibrated time period is obtained, where the multiples can data include a large amount of clinical data corresponding to multiple scans of the target system within the calibrated time period. Superposition processing is performed on the energy information in the scan data, and the energy distribution after superposition is the energy response within the calibrated time period. For example, the scan data accumulated in the recent week is superposed to obtain the energy distribution after superposition as the energy response within the calibrated time period. For another example, data is accumulated based on a preset number of scans, such as accumulating data every 50 scans. This embodiment does not limit the accumulation mode of clinical scan data.

In some embodiments, the average energy response of the human body of the system detector is used to reflect the change of the state of the target system, and the each of the multiple energy responses of the target system can be simplified as a preset function, including a Gaussian function, a double Gaussian function, a composite function composed of a Gaussian function and an exponential function, etc., so that the function formula corresponding to the system state is determined according to the detector's energy response curve of the average human body under each state of the target system, so as to parameterize the state of the target system.

In some embodiments, the preset function includes a Gaussian function, a double Gaussian function, or a composite function composed of the Gaussian function and the exponential function. Under a certain system state, template parameters such as the curve peak, standard deviation, and mean value in the energy response function formula are used to reflect the change of the system state. Based on this, the mapping relationship between the system state variation and the calibration parameters is obtained by constructing the mapping relationship between the plurality of groups of calibration parameters of the target system and the plurality of state variations of the target system and the current state variation of the target system.

In some embodiments, a reference energy response of the target system is obtained, which is the energy response information of the target system in a reference state. The reference state includes the system sensitivity calibrated by a sensitivity calibration phantom, such as the historical sensitivity of the system calibrated regularly, or the system sensitivity after the first calibration after the system is newly installed or a system failure occurs. It is determined whether the energy variation between the energy response within the calibrated time period and the reference energy response is greater than an energy threshold. if the energy variation is greater than the energy threshold, calibrating the sensitivity of the target system based on the variation of the energy response during the calibrated time period relative to the reference energy response, wherein the calibrating the sensitivity of the target system includes at least one of calibrating an energy window of the target system or calibrating a count rate of the target system; for example, the sensitivity of the target system needs to be calibrated when the variation of the energy response within the calibrated time period relative to the reference energy response exceeds 1 Kev. The calibration methods include but are not limited to calibrating the energy window of the target system, calibrating the count rate of the target system, or a combination thereof. If the energy variation between the energy response within the calibrated time period and the reference energy response is less than or equal to the energy threshold, that is, the system state variation is within the allowable range of the target system, so that the change of the actual sensitivity of the target system within the calibrated time period is also within the controllable range of the target system, and the system sensitivity in the reference state is used as the system sensitivity in the current state, that is, for the period between two sensitivity calibrations, the sensitivity after the last calibration is used as the reference to calibrate the sensitivity in the current state, so that there is no need to calibrate the sensitivity again, which realizes a significant extension of the calibration cycle, accurately calibrates the system sensitivity, and ensures that the calibrated system adapts to the continuous change of the state of the target system.

Through this embodiment, the energy response of the target system within the calibrated time period is obtained, the reference energy response of the target system is obtained, and it is determined whether the energy response within the current time period and the reference energy response meet the energy threshold. If the threshold is exceeded, the sensitivity of the target system is calibrated in response to the variation of the energy response within the current time period relative to the reference energy response, so as to realize accurate calibration of the target system by using the reference energy response of the system.

In some of these embodiments, calibrating the sensitivity of the target system in step 1040 includes step 1041 to step 1044.
Step 1041, determine a current state of the target system during the calibrated time period and a reference state of the target system, wherein the reference state corresponds to the reference energy response;
Step 1042, determine a current state variation of the target system relative to the reference state of the target system based on a comparison result of the energy response during the calibrated time period and the reference energy response;
Step 1043, determine a mapping relationship between a plurality of groups of calibration parameters of the target system and a plurality of state variations of the target system;
Step 1044, calibrating the sensitivity of the target system based on the mapping relationship between the plurality of groups of calibration parameters of the target system and the plurality of state variations of the target system and the current state variation of the target system, wherein the calibrating the sensitivity of the target system includes at least one of calibrating the energy window of the target system or calibrating the count rate of the target system.

In some embodiments, the current state of the target system within the calibrate time period and the reference state of the target system are determined, where the reference state refers to the state of the target system when the energy response of the target system is the reference energy response. The current state of the target system within the calibrate time period is compared with the reference energy response, and the current system state variation of the current state relative to the reference state is determined according to the comparison result.

In some embodiments, according to the mapping relationship between the plurality of groups of calibration parameters of the target system and the plurality of state variations of the target system and the current state variation of the target system, the calibration parameters corresponding to the current system state variation are determined, and the system is calibrated using the calibration parameters corresponding to the current system state variation. The calibration methods include at least calibrating the energy window of the target system or calibrating the count rate of the target system, etc.

In some embodiments, the energy window of the target system is calibrated using the calibration parameters corresponding to the current system state variation, including calibrating the energy window based on energy window parameters, which include the center position of the energy window, or the width of the energy window, etc., by setting the center position of the energy window, the width of the energy window, etc. In some embodiments, when calibrating the scaling factor of the target system, on the basis of keeping the energy window unchanged, a reasonable scaling factor is set according to the calibration parameters corresponding to the current system state variation, so as to realize the calibration of the count rate of the target system.

Through this embodiment, the current state of the target system within the calibrate time period and the reference state of the target system are determined, the current system state variation of the current state relative to the reference state is determined according to the comparison result of the energy response within the calibrate time period and the reference energy response, and then the mapping relationship between the calibration parameters of the target system and the system state variation is determined. The energy window of the target system and/or the count rate of the target system are calibrated based on the mapping relationship and the current system state variation, so that the system is calibrated according to the change of the current system state compared with the reference state, and the corresponding calibration method can be reasonably selected according to the actual application scenario.

The present embodiment is further described and explained through another embodiment.

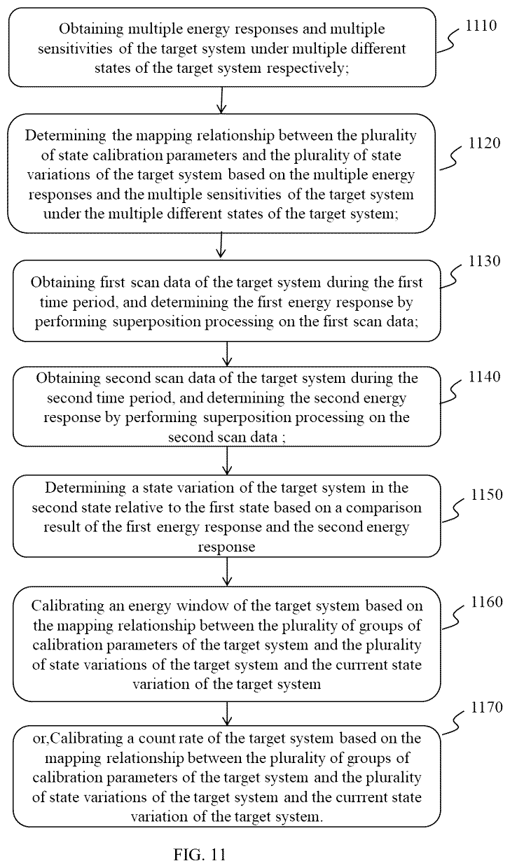

FIG. 11 is a flowchart of a sensitivity calibration method for a SPECT system in the present embodiment. As shown in FIG. 10, the sensitivity calibration method for the SPECT system includes step 1110 to step 1170.

Step 1110, obtaining multiple energy responses and multiple sensitivities of the target system under multiple different states of the target system respectively.

Step 1120, determining the mapping relationship between the plurality of state calibration parameters and the plurality of state variations of the target system based on the multiple energy responses and the multiple sensitivities of the target system under the multiple different states of the target system.

Step 1130, obtaining first scan data of the target system during the first time period, and determining the first energy response by performing superposition processing on the first scan data.

Step 1140, obtaining second scan data of the target system during the second time period, and determining the second energy response by performing superposition processing on the second scan data.

Step 1150, determining a state variation of the target system in the second state relative to the first state based on a comparison result of the first energy response and the second energy response.

Step 1160, calibrating an energy window of the target system based on the mapping relationship between the plurality of groups of calibration parameters of the target system and the plurality of state variations of the target system and the current state variation of the target system.

Step 1170, or, calibrating a count rate of the target system based on the mapping relationship between the plurality of groups of calibration parameters of the target system and the plurality of state variations of the target system and the current state variation of the target system.

Through the device provided in this embodiment, the energy responses and sensitivities of the target system under different system states are obtained, and the mapping relationship between the plurality of groups of calibration parameters of the target system and the plurality of state variations of the target system and the current state variation of the target system.

In some embodiments, within the first time period, first scan data of the target system in the first state is obtained, and the first energy response is obtained by superposing the first scan data. Within the second time period, second scan data of the target system in the second state is obtained, and the second energy response is obtained by superposing the second scan data, so that the current system state variation of the second state relative to the first state can be determined according to the comparison result of the first energy response and the second energy response, so as to accurately reflect the system state variation.

In some embodiments, the energy window of the target system is calibrated based on the mapping relationship and the current system state variation, or the count rate of the target system is calibrated based on the mapping relationship and the current system state variation, which solves the problem that the system sensitivity cannot be accurately calibrated to adapt to the continuous change of the system state, realizes accurate calibration of the system sensitivity, and ensures that the calibrated system adapts to the continuous change of the system state.

Another embodiment is described and illustrated below to further explain the present embodiment.

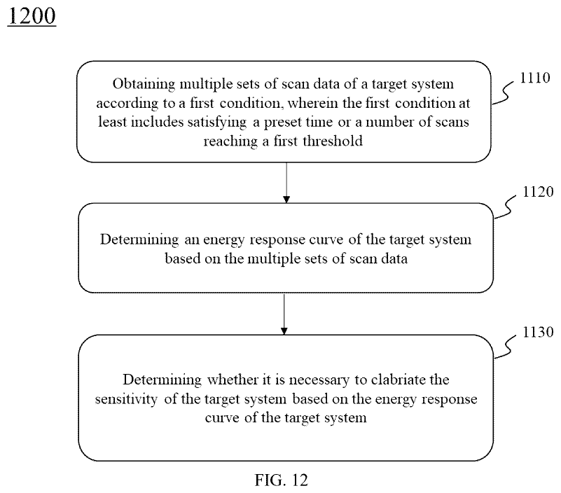

FIG. 12 is a flowchart of a sensitivity correction method for the SPECT system of the present embodiment. As shown in FIG. 12, the sensitivity correction method for the SPECT system includes step 1210 to step 1240.

Step 1210, obtain multiple sets of scan data of a target system according to a first condition, wherein the first condition at least includes satisfying a preset time or a number of scans reaching a first threshold.

In some embodiments, the multiple sets of scan data are data obtained by scanning the same part of different scanning objects with the target system, or scan data of various scanning objects with similar body mass indexes.

In some implementation examples, the first condition includes a preset time, such as scan data of the target system within a week. The first condition includes that the number of scans meets a first threshold; for example, when the number of scans reaches 50, multiple sets of scan data are acquired for analysis. Further, for example, the first condition includes that within a preset time and the cumulative number of scans reaches 50, multiple sets of scan data are acquired for analysis.

Step 1220, determine an energy response curve of the target system based on the multiple sets of scan data.

In some embodiments, when the system state changes, the number of photons received by the detector changes over a period of time, thereby causing a change in the energy response curve and a change in the sensitivity of the target system. Therefore, it can be determined whether the sensitivity of the target system needs to be corrected through the energy response curve.

In some embodiments, the acquired multiple sets of scan data are subjected to superimposition processing using properties of a Gaussian distribution to determine an energy response curve of a target form. The manner of acquiring the energy response curve has been described in the foregoing embodiments, and will not be repeated here.

S1230: determining whether it is necessary to calibrate the sensitivity of the target system based on the energy response curve of the target system.

In some embodiments, the energy response curve includes a peak value, i.e., an energy peak (for example, FIG. 3(a), 3(b), and FIG. 4). It is determined whether the sensitivity of the target system needs to be corrected by the change in the energy peak of the energy response curve. The manner of correcting the sensitivity of the target system has been described in the foregoing embodiments, and will not be repeated here.

In the present embodiment, the energy response curve of the target system is determined through multiple sets of scan data that meet the first condition, and a judgment on whether to perform sensitivity correction is made based on the energy response curve. This achieves a significant extension of the correction cycle, accurately corrects the sensitivity of the system, and ensures that the corrected system adapts to the continuous changes in the system state.

The present embodiment also provides a computer device, including a storage device 104 and a processor, where a computer program is stored in the storage device 104, and the processor 102 is configured to run the computer program to execute the steps in any of the above method embodiments.

Optionally, the above computer device further includes a transmission device 106 and an input/output device 108, where the transmission device is connected to the processor 102, and the input/output device 108 is connected to the processor 102.

Optionally, in this embodiment, the above processor may be configured to execute the following steps through a computer program.
S1, obtain a mapping relationship between a plurality of groups of calibration parameters of a target system and a plurality of state variations of the target system;
S2, obtain a first energy response of the target system during a first time period and a second energy response of the target system during a second time period; wherein the target system is in a first state during the first time period, and the target system is in a second state during the second time period.

S3, determine a state variation of the target system in the second state relative to the first state based on a comparison result of the first energy response and the second energy response.

S4, calibrating a sensitivity of the target system based on the mapping relationship between the plurality of groups of calibration parameters of the target system and the plurality of state variations of the target system and the current state variation of the target system.

It should be noted that the processes 300, 1000, 1100 and 1100 and the descriptions thereof are provided for the purposes of illustration, and not intended to limit the scope of the present disclosure. For persons having ordinary skills in the art, various modifications and changes in the forms and details of the application of the above method and system may occur without departing from the principles of the present disclosure. However, those variations and modifications also fall within the scope of the present disclosure. For example, the operations of the illustrated processes are intended to be illustrative. In some embodiments, the processes may be accomplished with one or more additional operations not described, and/or without one or more of the operations discussed. Additionally, the order in which the operations of the processes and regarding descriptions are not intended to be limiting.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “module,” “unit,” “component,” “device,” or “system”. Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an subject oriented programming language such as Java, Scala, Smalltalk, Eiffel, JADE, Emerald, C++, C#, VB. NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claim subject matter lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially”. For example, “about,” “approximate,” or “substantially” may indicate a certain variation (e.g., ±1%, ±5%, +10%, or ±20%) of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable. In some embodiments, a classification condition used in classification or determination is provided for illustration purposes and modified according to different situations. For example, a classification condition that “a value is greater than the threshold value” may further include or exclude a condition that “the probability value is equal to the threshold value”

What is claimed is:

1. A method for sensitivity calibration, comprising:
obtaining a mapping relationship between a plurality of groups of calibration parameters of a target system and a plurality of state variations of the target system;
obtaining a first energy response of the target system during a first time period and a second energy response of the target system during a second time period; wherein the target system is in a first state during the first time period, and the target system is in a second state during the second time period;
determining a state variation of the target system in the second state relative to the first state based on a comparison result of the first energy response and the second energy response; and
calibrating a sensitivity of the target system based on the mapping relationship between the plurality of groups of calibration parameters of the target system and the plurality of state variations of the target system and the current state variation of the target system.

2. The method of claim 1, wherein the obtaining the mapping relationship between the plurality of groups of calibration parameters of the target system and the plurality of state variations comprises:
obtaining multiple energy responses and multiple sensitivities of the target system under multiple different states of the target system respectively, wherein one of the multiple energy responses and one of the multiple sensitivities of the target system correspond to one of the multiple different states of the target system; and
determining the mapping relationship between the plurality of state calibration parameters and the plurality of state variations of the target system based on the multiple energy responses and the multiple sensitivities of the target system under the multiple different states of the target system.

3. The method of claim 2, wherein each of the multiple energy responses of the target system is represented by a preset function.

4. The method of claim 3, wherein the preset function comprises a Gaussian function, a double Gaussian function, or a composite function composed of the Gaussian function and the exponential function.

5. The method of claim 3, wherein the mapping relationship is a relationship between variations of one or more target parameters in the preset function and the plurality of groups of calibration parameters.

6. The method of claim 2, wherein the mapping relationship between the plurality of groups of calibration parameters of the target system and the plurality of state variations of the target system comprises:
selecting the mapping relationship between the plurality of groups of calibration parameters of the target system and the plurality of state variations of the target system from a preset mapping relationship library based on a combination of isotope types and collimator types.

7. The method of claim 1, wherein the obtaining the first energy response of the target system during the first time period and the second energy response of the target system during the second time period comprises:
obtaining first scan data of the target system during the first time period, and determining the first energy response by performing superposition processing on the first scan data; and
obtaining second scan data of the target system during the second time period, and determining the second energy response by performing superposition processing on the second scan data.

8. The method of claim 1, wherein the calibrating the sensitivity of the target system based on the mapping relationship between the plurality of groups of calibration parameters of the target system and the plurality of state variations of the target system and the current state variation of the target system comprises:
determining one group of calibration parameters corresponding to the state variations of the target system based on the mapping relationship between the plurality of groups of calibration parameters of the target system and the plurality of state variations of the target system and the current state variation of the target system, wherein the one group calibration parameters include at least one of energy window parameters or scale factor.

9. The method of claim 8, wherein the calibrating the sensitivity of the target system based on the mapping relationship between the plurality of groups of calibration parameters of the target system and the plurality of state variations of the target system and the current state variation of the target system comprises:
calibrating an energy window of the target system based on the mapping relationship between the plurality of groups of calibration parameters of the target system and the plurality of state variations of the target system and the current state variation of the target system.

10. The method of claim 9, wherein the calibrating the sensitivity of the target system based on the mapping relationship between the plurality of groups of calibration parameters of the target system and the plurality of state variations of the target system and the current state variation of the target system comprises:
determining the energy window parameters based on the mapping relationship between the plurality of groups of calibration parameters of the target system and the plurality of state variations of the target system and the current state variation of the target system, wherein the energy window parameters at least include a center position of the energy window or a width of the energy window; and
calibrating the energy window of the target system based on the energy window parameters.

11. The method of claim 8, wherein the calibrating the sensitivity of the target system based on the mapping relationship between the plurality of groups of calibration parameters of the target system and the plurality of state variations of the target system and the current state variation of the target system comprises:
calibrating a count rate of the target system based on the mapping relationship between the plurality of groups of calibration parameters of the target system and the plurality of state variations of the target system and the current state variation of the target system.

12. The method of claim 11, wherein the calibrating the count rate of the target system based on the mapping relationship between the plurality of groups of calibration parameters of the target system and the plurality of state variations of the target system and the current state variation of the target system comprises:
determining a scale factor of the target system based on the mapping relationship between the plurality of groups of calibration parameters of the target system and the plurality of state variations of the target system and the current state variation of the target system, when an energy window corresponding to the first state and an energy window corresponding to the second state of the target system are the same; and
calibrating the count rate of the target system based on the scale factor of the target system.

13. The method of claim 12, wherein the determining the scale factor of the target system based on the mapping relationship between the plurality of groups of calibration parameters of the target system and the plurality of state variations of the target system and the current state variation of the target system comprises:
constructing a mapping relationship between multiple energy drifts and multiple scale factors; and
determining the scale factor corresponding to the current state variation of the target system based on the mapping relationship between the multiple energy drifts and multiple scale factors.

14. A method for sensitivity calibration, comprising:
obtaining an energy response of a target system during a calibrated time period;
obtaining a reference energy response of the target system;
determining whether an energy variation between the energy response during the calibrated time period and the reference energy response is greater than an energy threshold; and
if the energy variation is greater than the energy threshold, calibrating the sensitivity of the target system based on the variation of the energy response during the calibrated time period relative to the reference energy response, wherein the calibrating the sensitivity of the target system includes at least one of calibrating an energy window of the target system or calibrating a count rate of the target system.

15. The method of claim 14, wherein the calibrating the sensitivity of the target system comprises: determining a current state of the target system during the calibrated time period and a reference state of the target system, wherein the reference state corresponds to the reference energy response; determining a current state variation of the target system relative to the reference state of the target system based on a comparison result of the energy response during the calibrated time period and the reference energy response;
determining a mapping relationship between a plurality of groups of calibration parameters of the target system and a plurality of state variations of the target system; and
calibrating the sensitivity of the target system based on the mapping relationship between the plurality of groups of calibration parameters of the target system and the plurality of state variations of the target system and the current state variation of the target system, wherein the calibrating the sensitivity of the target system includes at least one of calibrating the energy window of the target system or calibrating the count rate of the target system.

16. The method of claim 15, wherein the calibrating the sensitivity of the target system includes at least one of the energy window of the target system or the count rate of the target system based on the mapping relationship the plurality of groups of calibration parameters of the target system and the plurality of state variations of the target system and the current state variation of the target system comprises:
determining energy window parameters based on the mapping relationship between the plurality of groups of calibration parameters of the target system and the plurality of state variations of the target system and the current state variation of the target system, wherein the energy window parameters include at least one of a center position of the energy window or a width of the energy window; and
calibrating the energy window of the target system based on the energy window parameters.

17. The method of claim 16, wherein the calibrating the sensitivity of the target system includes at least one of the energy window of the target system or the count rate of the target system based on the mapping relationship between the plurality of groups of calibration parameters of the target system and the plurality of state variations of the target system and the current state variation of the target system comprises:
determining a scale factor of the target system based on the mapping relationship between the plurality of groups of calibration parameters of the target system and the plurality of state variations of the target system and the current state variation of the target system, when an energy window corresponding to the current state and an energy window corresponding to the reference state of the target system are the same; and
calibrating the count rate of the target system based on the scale factor of the target system.

18. The method of claim 17, wherein the determining the scale factor of the target system based on the mapping relationship between the plurality of groups of calibration parameters of the target system and the plurality of state variations of the target system and the current state variation of the target system comprises:
constructing a mapping relationship between multiple energy drifts and the multiple scale factors; and
determining the scale factor of the target system corresponding to the current state variation of the target system based on the mapping relationship between the multiple energy drifts and the multiple scale factors.

19. The method of claim 14, further comprising:
if the energy variation between the energy response during the calibrated time period and the reference energy response is less than or equal to the energy threshold, taking the sensitivity of the target system in the reference state as the sensitivity of the target system in the current state.

20. A method for sensitivity calibration, comprising:
obtaining multiple sets of scan data of a target system according to a first condition, wherein the first condition at least includes satisfying a preset time or a number of scans reaching a first threshold;
determining an energy response curve of the target system based on the multiple sets of scan data; and
determining whether it is necessary to calibrate the sensitivity of the target system based on the energy response curve of the target system.
